(19) United States
(12) Patent Application Publication
(10) Pub. No.: US 2023/0084433 A1
(43) Pub. Date: Mar. 16, 2023

(54) ENDOSCOPE

(71) Applicant: OLYMPUS MEDICAL SYSTEMS CORP., Tokyo (JP)

(72) Inventor: Keisuke HATANO, Tokyo (JP)

(73) Assignee: OLYMPUS MEDICAL SYSTEMS CORP., Tokyo (JP)

(21) Appl. No.: 17/475,731

(22) Filed: Sep. 15, 2021

(51) Int. Cl. A61B 1/005

(52) U.S. Cl. CPC A61B 1/0057; A61B 1/0052

(57) ABSTRACT

An endoscope according to an embodiment includes an elongated insertion section including a bending section configured to bend in at least an up-down direction by internally-disposed two or more wires being towed, an operation section including a grasping section, the operation section, a bending operation unit including a joystick for bending the bending section and provided in the operation section, two actuators respectively including turning shafts, the two turning shafts being internally disposed in the grasping section in a longitudinal direction of the grasping section, the two actuators being disposed side by side along a plane parallel to an operation direction of an operation member for bending the bending section in the up-down direction, and a conversion mechanism including a rotation torque transmission mechanism and a tensile force transmission mechanism, the conversion mechanism converting the rotation torque into the tensile force.

### BACKGROUND OF THE INVENTION

### 1. Field of the Invention

The present invention relates to an endoscope in which a bending operation of a bending section is possible by an actuator.

### 2. Description of Related Art

An endoscope is widely used in a medical field and an industrial field. The endoscope includes an elongated insertion section, and an observation optical system is provided at a distal end portion of the insertion section. Further, some endoscope includes a bending section in a distal end side portion of the insertion section. The bending section enables the distal end portion of the insertion section to bend in upward and downward two directions or upward, downward, left, and right four directions. By providing the bending section in the insertion section, it is possible to achieve improvement of insertability into a subject and change a visual field direction to perform a wide-range observation.

An operation member provided in an operation section is operated by a user of the endoscope, whereby bending of the bending section is performed. For example, International Publication No. 2016/147457 discloses an endoscope in which an operation member is, for example, a joystick. In the endoscope, proximal ends of four wires inserted through an insertion section are fixed to four arm members fixed to a proximal end portion of the joystick. Distal ends of the respective wires are connected to predetermined positions of bending pieces of a bending section. A user tilts the joystick, whereby the four wires inserted through the insertion section are towed and slacked and the bending section bends.

There has also been proposed an endoscope in which a towing and slacking operation of the four wires is performed using an actuator such as a motor. For example, Japanese Patent Application Laid-Open Publication No. 2013-158612 discloses an endoscope in which four wires are towed using a motor.

### SUMMARY OF THE INVENTION

An endoscope according to an aspect of the present invention includes: an elongated insertion section including a bending section configured to bend in at least an up-down direction by internally-disposed two or more strip-shaped members being towed; an operation section including a grasping section grasped by a hand, the operation section being disposed on a proximal end side of the insertion section; a bending operation unit including an operation member for bending the bending section and provided in the operation section; two actuators each including turning shafts, the two turning shafts being internally disposed in the grasping section in a longitudinal direction of the grasping section, the two actuators being disposed side by side along a plane parallel to an operation direction of the operation member for bending the bending section in the up-down direction; and a conversion mechanism including a rotation torque transmission mechanism to which rotation torque of the two turning shafts is transmitted and a tensile force transmission mechanism configured to transmit a tensile force for pulling the two or more strip-shaped members, the conversion mechanism converting the rotation torque into the tensile force.

### BRIEF DESCRIPTION OF THE DRAWINGS

FIG. 1 is a configuration diagram showing a configuration of an endoscope apparatus according to an embodiment of the present invention:

FIG. 2 is a sectional view of a distal end portion and a bending section according to the embodiment of the present invention:

FIG. 3 is a front view of an operation section according to the embodiment of the present invention:

FIG. 4 is a sectional view taken in a longitudinal direction of the operation section according to the embodiment of the present invention;

FIG. 5 is a sectional view of the operation section taken along a V-V line in FIG. 4;

FIG. 6 is a front view of a driving unit provided in the operation section according to the embodiment of the present invention;

FIG. 7 is a perspective view of the driving unit provided in the operation section according to the embodiment of the present invention,

FIG. 8 is a schematic configuration diagram of an operation section showing disposition of actuators in the operation section according to a modification 1 of the embodiment of the present invention;

FIG. 9 is a schematic configuration diagram of the operation section showing the disposition of the actuators in the operation section according to the modification 1 of the embodiment of the present invention;

FIG. 10 is a schematic configuration diagram of an operation section showing disposition of actuators in the operation section according to a modification 2 of the embodiment of the present invention; and

FIG. 11 is a schematic configuration diagram of the operation section showing the disposition of the actuators in the operation section according to the modification 2 of the embodiment of the present invention.

### DETAILED DESCRIPTION OF PREFERRED EMBODIMENT

An embodiment of the present invention is explained below with reference to the drawings.

### Configuration of an Endoscope Apparatus

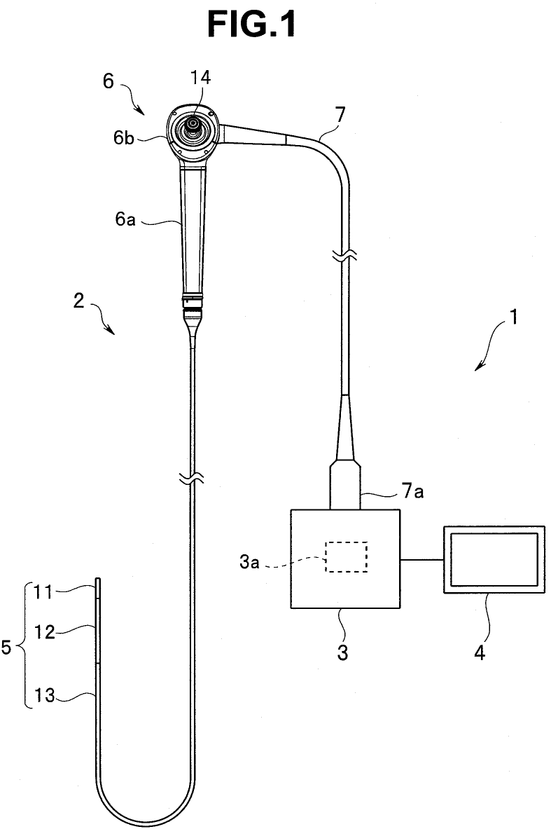

FIG. 1 is a configuration diagram showing a configuration of an endoscope apparatus according to the present embodiment. An endoscope apparatus 1 includes an endoscope 2, a main body apparatus 3. and a monitor 4.

The endoscope 2 includes an elongated insertion section 5, an operation section 6, and a connection cord 7. The connection cord 7 extends from a side portion of the operation section 6, and various signal lines and the like are inserted through the connection cord 7. The connection cord 7 includes a connector 7a at one end. The connector 7a is configured to be connectable to a connector (not shown) of the main body apparatus 3.

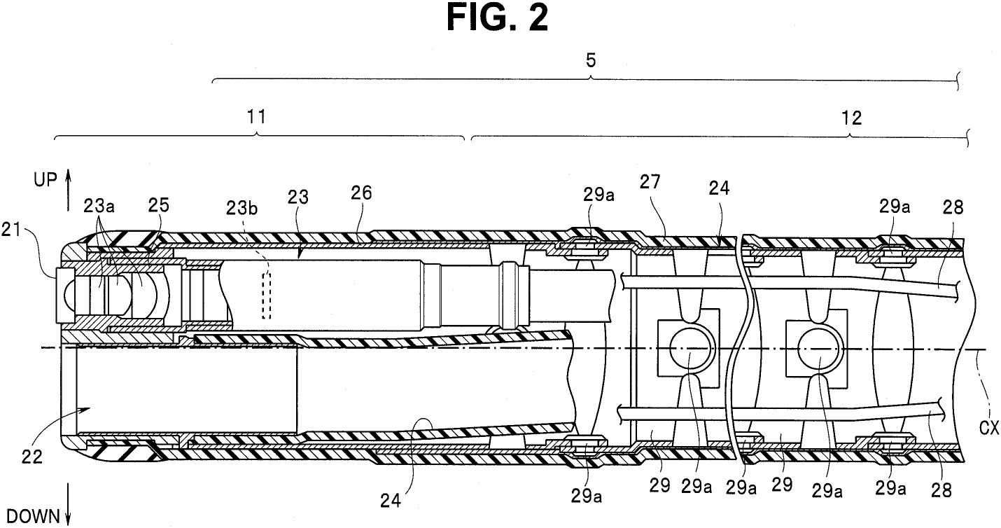

The insertion section 5 includes a distal end portion 11, a bending section 12, and a flexible tube section 13 in order from a distal end. The distal end portion 11 includes an observation window 21 (FIG. 2) and an illumination window. illumination light is emitted from the illumination window, and reflected light from a subject is received by an image pickup device 23b (FIG. 2) in the distal end portion 11 through the observation window 21.

The operation section 6 is disposed on a proximal end side of the insertion section 5. The operation section 6 includes a grasping section 6a to be grasped by a surgeon, who is a user of the endoscope 2, with one hand and an operation main body section 6b provided on a proximal end side (an upper side in FIG. 1) of the grasping section 6a. The grasping section 6a is a portion grasped by a hand of the surgeon. A bending operation unit 36 includes a joystick 14 functioning as an operation member for bending the bending section 12 and is provided in the operation main body section 6b. The joystick 14 is an operation member for bending the bending section 12 in an up-down direction and a left-right direction.

The insertion section 5 is inserted into the subject from the distal end portion 11. For example, the surgeon, who is the user of the endoscope 2, holds the insertion section 5 with a right hand and performs advancing and retracting operation to and from an inside of the subject and grasps the grasping section 6a with a left hand, operates the joystick 14 with a thumb of the left hand, and operates various operation buttons with other fingers.

The main body apparatus 3 is a video processor that controls an operation of the entire endoscope apparatus 1 and performs image processing for generating an endoscopic image. The main body apparatus 3 includes a processor 3a for operation control and image generation processing. A signal of the generated endoscopic image is outputted to the monitor 4 connected to the main body apparatus 3.

For example, when the surgeon presses a recording button of the operation main body section 6b, a signal indicating that the recording button is pressed is transmitted to the processor 3a. The processor 3a stores, based on the signal, a movie or a still image of the endoscopic image in a storage apparatus (not shown).

Accordingly, the surgeon can perform inspection of the inside of the subject by, for example, observing the inside of the subject and recording a necessary endoscopic image while viewing the endoscopic image displayed on the monitor 4.

### Configuration of the Distal End Portion of the Insertion Section

FIG. 2 is a sectional view of the distal end portion 11 and the bending section 12. The observation window 21, the illumination window (not shown), and an opening 22 are provided on a distal end face of the distal end portion 11. The opening 22 is a distal end side opening of a treatment instrument insertion channel 24 provided in the insertion section 5. A treatment instrument and the like can be inserted through the treatment instrument insertion channel 24, the surgeon is capable of projecting and retracting a distal end portion of the treatment instrument from the opening 22.

An image pickup unit 23 is incorporated in the distal end portion 11. The image pickup unit 23 includes a lens group 23a and the image pickup device 23b. A plurality of signal lines extending from the image pickup device 23b are electrically connected to the main body apparatus 3 through the insertion section 5, the operation section 6, and the connection cord 7.

The distal end portion 11 includes a distal end rigid member 25, the image pickup unit 23 is fixed to the distal end rigid member 25.

A distal end of the bending section 12 is connected to a proximal end of the distal end portion 11. In the distal end portion 11, a substantially cylindrical distalmost bending piece 26 configuring the bending section 12 is fixed to the proximal end side of the distal end rigid member 25. An outer circumference of the distalmost bending piece 26 is covered by bending rubber 27. Four wire fixing sections (not shown) are provided in a circumferential direction on an inner circumference of the distalmost bending piece 26. A distal end of one wire 28 inserted through the insertion section 5 is fixed to the respective wire fixing sections. Proximal ends of four wires 28 reach an inside of the operation section 6.

On the proximal end side of the distalmost bending piece 26, a plurality of bending pieces 29 are consecutively connected along a longitudinal axis CX of the insertion section 5. Each of the bending pieces 29 is connected to an adjacent bending piece 29 by two rivets 29a. More specifically, the plurality of bending pieces 29 are coupled such that two bending pieces 29 adjacent to each other along the longitudinal axis CX are turnable around an axis formed by the two rivets 29a In other words, a turning axis of the two bending pieces 29 adjacent to each other is defined by the two rivets 29a. The plurality of bending pieces 29 are turnably coupled by a plurality of rivets 29a along the longitudinal axis CX such that two turning shafts adjacent to each other are different by 90° in a circumference direction.

In this way, the bending section 12 bends in the up-down direction and the left-right direction by towing the wires 28, which are internally-disposed strip-shaped members.

### Configuration of the Operation Section

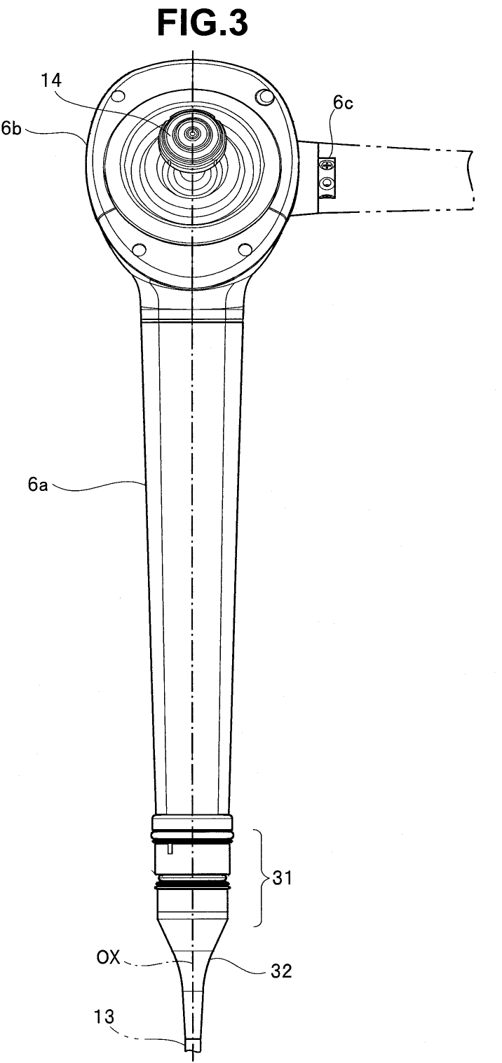

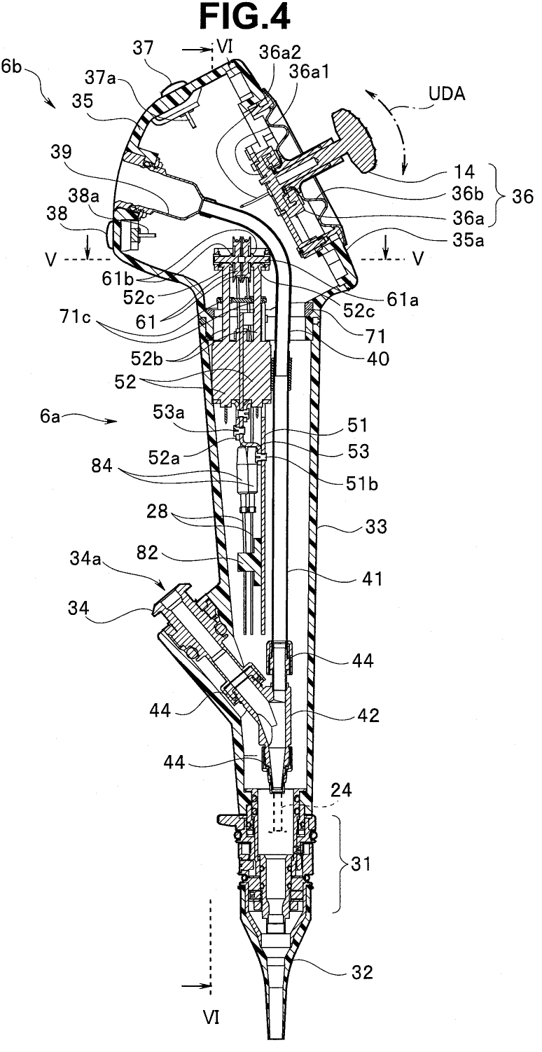

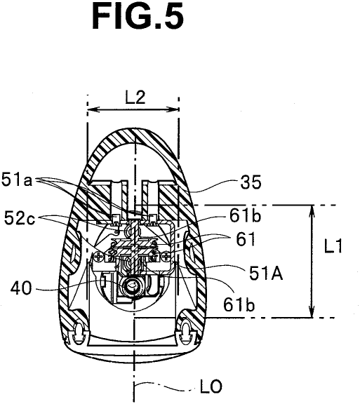

FIG. 3 is a front view of the operation section 6. FIG. 4 is a sectional view taken in a longitudinal direction of the operation section 6. FIG. 5 is a sectional view of the operation section 6 taken along a V-V line in FIG. 4.

As explained above, the operation section 6 includes the grasping section 6a and the operation main body section 6b. A connecting section 31, to which a proximal end of the insertion section 5 is connected, is provided at a distal end of the grasping section 6a. A bend preventing member 32 made of resin covering a part of the connecting section 31 and a proximal end portion of the insertion section 5 is provided at a distal end of the connecting section 31.

The grasping section 6a includes a tubular exterior member 33 made of resin. The grasping section 6a has a shape slightly thinned from the operation main body section 6b side toward the connecting section 31 side.

As shown in FIG. 5. a cross section of the grasping section 6a orthogonal to a longitudinal axis OX (FIG. 3) of the grasping section 6a has a substantially partially elliptical shape. The cross section of the grasping section 6a has a shape extending in a direction of a longitudinal axis LO of a substantially partial ellipse.

in other words, the grasping section 6a has a shape extending in a longitudinal direction (a direction of the longitudinal axis OX), and a sectional shape of the grasping section 6a orthogonal to the longitudinal direction has anisotropy.

When bending the bending section 12 in the up-down direction, the surgeon moves the joystick 14 in a direction indicated by an arrow UDA of an alternate long and short dash line in FIG. 4. Accordingly, as shown in FIG. 5, in the sectional shape orthogonal to the longitudinal direction of the grasping section 6a, a dimension L1 in a direction parallel to a plane parallel to an operation direction of the joystick 14 for bending the bending section 12 in the up-down direction is larger than a dimension L2 in a direction perpendicular to the plane.

As shown in FIG. 4. the grasping section 6a includes a treatment instrument insertion section 34. The treatment instrument insertion section 34 includes a treatment instrument insertion opening 34a into which various treatment instruments (not shown) can be inserted. The treatment instrument insertion opening 34a is a proximal end side opening of the treatment instrument insertion channel 24.

The operation main body section 6b includes an exterior member 35 fixed to a proximal end portion (a portion on an upper side in FIG. 4) of the exterior member 33. The bending operation unit 36 including the joystick 14 is disposed in and fixed to. via a cover member 35a, an opening provided in a part of the exterior member 35.

The bending operation unit 36 includes the joystick 14, which is an operation member, a detection apparatus 36a fixed to the cover member 35a, and a skirt member 36b covering a lower part periphery of the joystick 14. The cover member 35a is a member fixed to the exterior member 35 to cover a part of the exterior member 35. The detection apparatus 36a is provided at a proximal end portion of the joystick 14 and includes a circuit board 36a1 that detects a tilting direction and a tilting angle in a tilting operation of the joystick 14 and outputs a tilting signal including information concerning the detected direction (tilting direction) and the detected angle (tiling angle). One end of a signal cable 36a2 is connected to the circuit board 36a1. The signal cable 36a2 is inserted through the connection cord 7. The other end of the signal cable 36a2 is electrically connected to the processor 3a of the main body apparatus 3. The signal cable 36a2 includes a plurality of signal lines for transmitting the tilting signal indicating the tilting direction and the tilting angle of the joystick 14 and a control signal from the processor 3a

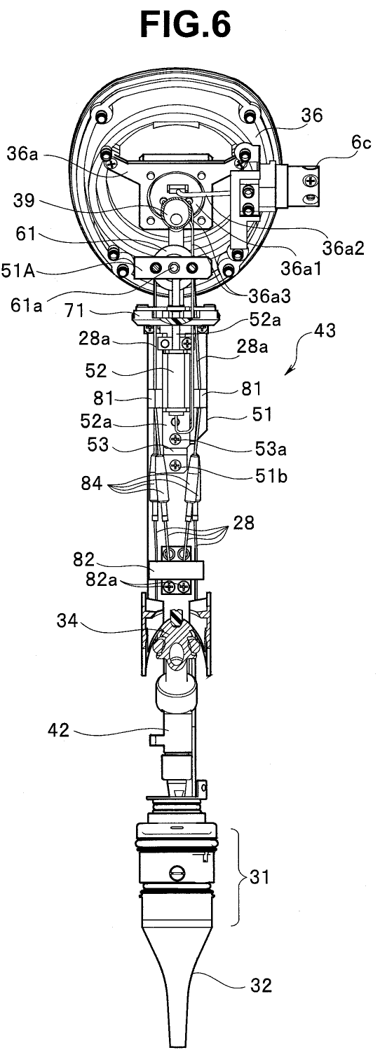

Further, a plurality of signal lines 36a3 (FIG. 6) also extend from the circuit board 36a1. One ends of the respective signal lines 36a3 are connected to the circuit board 36a1. The other ends of the respective signal lines 36a3 are connected to one motor 52. The circuit board 36a1 outputs, via the plurality of signal lines 36a3, driving signals for driving two motors 52 to the two motors 52. The respective motors 52 are actuators that operate according to a control signal from the circuit board 36a1.

The tilting signal of the joystick 14 is transmitted to the processor 3a of the main body apparatus 3 via the signal cable 36a2. The processor 3a generates, based on the tilting signal, a control signal for controlling operations of the two motors 52 and outputs the control signal to the circuit board 36a1 via a control signal line included in the signal cable 36a2. The circuit board 36a1 generates, based on the received control signal, driving signals for driving the respective motors 52 and outputs the driving signals to a plurality of motors 52 via the plurality of signal lines 36a3.

Note that a plurality of signal lines for driving the plurality of motors 52 may be inserted through the connection cord 7, the processor 3a and the plurality of motors 52 may be directly connected. In that case, the processor 3a generates, based on the tilting signal received from the circuit board 36a1. driving signals for driving the respective motors 52 and outputs the driving signals to the respective motors 52.

The bending operation unit 36 is provided on an opposite side of the treatment instrument insertion section 34 with respect to the longitudinal axis OX of the grasping section 6a. More specifically, as shown in FIG. 4, when the operation section 6 is viewed to face the joystick 14 of the bending operation unit 36, the treatment instrument insertion section 34 is provided on a distal end side (a lower side in FIG. 4) of the grasping section 6a on an opposite side (a left side in FIG. 4) of the joystick 14 with respect to the longitudinal axis OX.

When bending the bending section 12 in the up-down direction, the surgeon moves the joystick 14 in the direction indicated by the arrow UDA of the alternate long and short dash line in FIG. 4. Accordingly, a movement of a distal end portion of the joystick 14 at the time when the bending section 12 is bent in the up-down direction is a movement in a plane including the longitudinal axis LO of the cross section of the grasping section 6a.

When bending the bending section 12 in the left-right direction, the surgeon tilts the joystick 14 in a direction orthogonal to the direction indicated by the arrow UDA of the alternate long and short dash line in FIG. 4. In other words, a movement of the distal end portion of the joystick 14 at the time when the bending section 12 is bent in the left-right direction is a movement in a direction orthogonal to the longitudinal axis LO of the cross section of the grasping section 6a.

Two button switches 37 and 38 are provided in the operation main body section 6b. and the respective button switches 37 and 38 are switches to which any functions are allocated out of various functions concerning the endoscope apparatus 1. As shown in FIG. 4, the two button switches 37 and 38 are provided on an opposite side of the bending operation unit 36 with respect to the longitudinal axis OX of the grasping section 6a. In other words, the two button switches 37 and 38 are provided on the same side as the treatment instrument insertion section 34 with respect to the longitudinal axis OX of the grasping section 6a. The two button switches 37 and 38 are disposed at an interval in the direction of the longitudinal axis OX of the grasping section 6a on the exterior member 35.

Two signal lines 37a and 38a extending from the two button switches 37 and 38 are electrically connected to the processor 3a of the main body apparatus 3 through the connection cord 7.

Further, a cylinder 39. to which a suction button (not shown) is detachably attachable, is provided in the operation main body section 6b. The surgeon can perform suction of blood and the like from the opening 22 of the distal end portion 11 of the insertion section 5 by pressing the suction button attached to the cylinder 39. One end of a suction tube 40 is connected to the cylinder 39. The other end of the suction tube 40 is connected to a branch connector 42 via another tube 41.

The branch connector 42 includes an internal channel branching in a Y shape. An opening on the insertion section 5 side of the branch connector 42 communicates with the treatment instrument insertion channel 24. Further, in the branch connector 42, the treatment instrument insertion opening 34a and the treatment instrument insertion channel 24 communicate. Further, the cylinder 39 and the treatment instrument insertion channel 24 also communicate. Each of the treatment instrument insertion section 34, the tube 41, and the treatment instrument insertion channel 24 is connected to the branch connector 42 by a connection member 44 such as a pipe sleeve.

A connecting section 6c for connection to the connection cord 7 is provided on a side surface of the operation main body section 6b.

In the operation section 6, a driving unit 43 including two motors for towing and slacking the four wires 28 and bending the bending section 12 is incorporated.

### Configuration of the Driving Unit

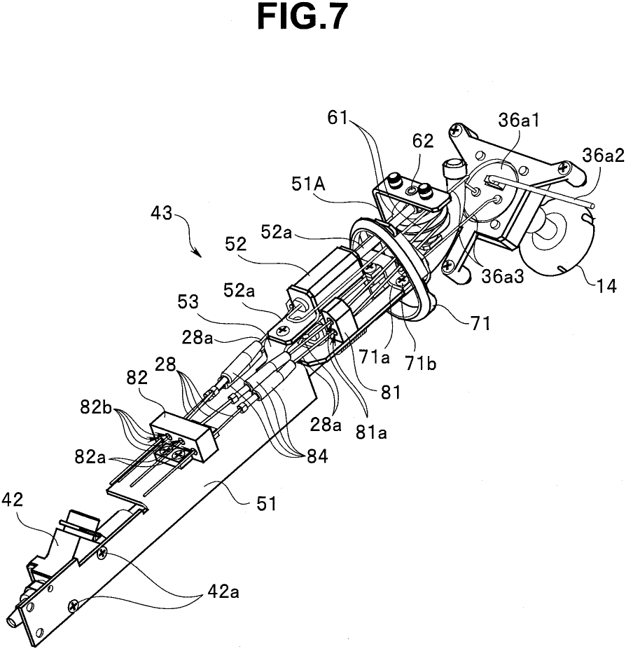

FIG. 6 is a front view of the driving unit 43 provided in the operation section 6. FIG. 7 is a perspective view of the driving unit 43 provided in the operation section 6. FIG. 6 is a view of the bending operation unit 36 and the driving unit 43 viewed from a rear side of the bending operation unit 36.

The driving unit 43 includes a support plate 51. The support plate 51 is made of metal such as stainless steel and has a shape obtained by partially cutting an elongated plate member and bending various portions of the plate member. The support plate 51 is fixed to the exterior member 35 by screws 51a (FIG. 5).

A fixing plate 53 for supporting and fixing the two motors 52 is fixed to the support plate 51. A latch plate 52a for the respective motors 52 is fixed to the fixing plate 53 by a screw 53a, whereby the two motors 52 are fixed to the fixing plate 53. The fixing plate 53 has a shape bent in a crank shape, and one end of the fixing plate 53 is fixed to the support plate 51 by a screw 51b. The two motors 52 are fixed to the other end of the fixing plate 53 to sandwich the fixing plate 53.

As shown in FIG. 4. the two motors 52 are disposed on an upper side (that is, a side close to the joystick 14) of the grasping section 6a such that, when the surgeon grasps the grasping section 6a. the two motors 52 are located on an inner side of a palm of a hand grasping the grasping section 6a.

One end side portion (an upper side portion of FIG. 6) of the support plate 51 is a pulley fixing section 51A. The pulley fixing section 51A is bent in an angular U shape. Two pulley shafts 61a of two pulleys 61 are fixed to the pulley fixing section 51A. The two pulley shafts 61a are disposed to be monoaxial between two plate sections formed by being bent in a U shape. The respective pulleys 61 are provided in the pulley fixing section 51A of the support plate 51 to be turnable around the pulley shafts 61a The respective pulley shafts 61a include bevel gears 61b, which are turning members. The respective pulleys 61 are made of metal such as stainless steel. The respective pulleys 61 include pulley grooves in outer circumferential portions, and wires 28a are laid in the pulley grooves and are towed by a tensile force by the turning.

Bevel gears 52c. which are turning members, are provided at distal end portions of turning shafts 52b of the respective motors 52. The bevel gears 52c turn according to the turning of the turning shafts 52b. The respective motors 52 and the respective pulleys 61 are disposed such that the turning shafts 52b of the respective motors 52 are orthogonal to the pulley shafts 61a of the respective pulleys 61 and the respective bevel gears 61b screw with one bevel gear 52c. The respective turning shafts 52b and the respective bevel gears 52c are made of metal such as stainless steel.

A fixing member 71 that turnably fixes the two turning shafts 52b of the two motors 52 is fixed to the support plate 51. A latch member 71a (FIG. 7) is fixed to the fixing member 71, and the latch member 71a is fixed to the support plate 51 by screws 71b.

The fixing member 71 includes two bearings 71c (FIG. 4) that support the two turning shafts 52b. The respective turning shafts 52b are inserted through holes of the bearings 71c to thereby be fixed to the support plate 51 to be turnable.

The respective bevel gears 52c turning according to the turning of the turning shafts 52b of the respective motors 52 are screwed with the bevel gears 61b of the pulleys 61. Therefore, when the turning shafts 52b of the respective motors 52 turn, the pulleys 61 also turn.

As explained above, the respective motors 52. which are the actuators, include the turning shafts 52b. The two turning shafts 52b of the two motors 52 are internally disposed in the grasping section 6a in the direction of the longitudinal axis OX of the grasping section 6a. The two motors 52 are disposed side by side along the plane parallel to the operation direction (the direction indicated by the arrow UDA) of the joystick 14 for bending the bending section 12 in the up-down direction.

The bevel gears 52c provided in the respective turning shafts 52b and the bevel gears 61b provided in the pulleys 61 configure a rotation torque transmission mechanism to which rotation torque of the turning shafts 52b is transmitted. As shown in FIG. 4, the four bevel gears 52c and 61b configuring the rotation torque transmission mechanism are disposed on the bending operation unit 36 side in the operation section 6. One bevel gear 52c and one bevel gear 61b screwing with the bevel gear 52c configure one torque conversion mechanism. The respective motors 52 transmit rotation torque to the one torque conversion mechanism. The operation section 6 includes two torque conversion mechanisms.

In one of the two pulleys 61, one end of each of two wires 28a for up-down bending is fixed in the pulley groove. In the other of the two pulleys 61, one end of each of the two wires 28a for left-right bending is fixed in the pulley groove. The two wires 28a are fit in the grooves of the pulleys 61 such that one of the two wires 28a is towed and the other of the two wires 28a is slacked by the turning of the respective pulleys 61.

When the respective pulleys 61 turn in one direction, the respective pulleys 61 tow one of the two wires 28a and slack the other. When the respective pulleys 61 turn in the opposite direction, the respective pulleys 61 slack one of the two wires 28a and tow the other.

Accordingly, the pulleys 61, in which the respective bevel gears 61b are provided, configure a tensile force transmission mechanism that transmits a tensile force for pulling the wires 28a. The four bevel gears 52c and 61b and the two pulleys 61 configure a conversion mechanism that converts rotation torque of the two motors 52 into a tensile force for the two wires 28a.

Two guide members 81 are fixed to the support plate 51 by screws (not shown) to sandwich the two motors 52. Each of the guide members 81 includes two holes 81a. One wire 28a is inserted through one hole 81a. Accordingly, as shown in FIGS. 6 and 7, four wires 28a are inserted through four holes 81a of the two guide members 81 fixed to the support plate 51.

A guide member 82 is also fixed to the support plate 51 by screws 82a. Proximal end portions of the four wires 28 inserted through the insertion section 5 are inserted through four holes 82b formed in the guide member 82. One wire 28 is inserted through one hole 82b.

Proximal ends of the four wires 28 and distal ends of the four wires 28a are connected by four coupling members 84 between the two guide members 81 and the guide member 82. The respective coupling members 84 also include mechanisms that connect the proximal ends of the wires 28 and the distal ends of the wires 28a and adjust length between the two wires 28 and 28a.

The branch connector 42 is fixed by screws 42a on an opposite side of the pulley fixing section 51A of the support plate 51.

### Effects

In general, operation of an operation member such as a joystick is performed by a finger (for example, a thumb) of one hand (for example, a left hand) of a user who grasps the operation section 6 of the endoscope 2. Accordingly, when towing of the respective wires 28 is performed by a mechanical mechanism, if an operation amount of force in tilting operation of the joystick increases, a large load is applied to the hand or the finger of the user to be a burden for the user.

In the case of the joystick, an operation amount of force for the joystick is determined according to a bending angle or the like of the bending section 12. In order to reduce the operation amount of force, it is necessary to increase a size of an arm member to which proximal ends of the respective wires are connected. However, if the arm member increases in size, a size of the operation section 6 itself increases. If the size of the operation section 6 itself increases, it is difficult to grasp the operation section 6. and operability for the user is thus lowered.

Therefore, in order to tow the respective wires with electric means, actuators such as motors can be used. However, depending on a way of disposition of two actuators in the operation section 6, the size of the operation section 6 increases, the user cannot stably grasp the operation section 6 with one hand, and the operability of the operation section 6 is lowered.

In contrast, according to the embodiment explained above, since the two motors 52 having a relatively large weight are disposed side by side in the longitudinal axis LO direction in the grasping section 6a, the grasping section 6a does not increase in size, the grasping section 6a can be stably grasped, and the user can easily grasp the grasping section 6a. As a result, the operability of the operation section 6 is high for the surgeon.

In particular, as shown in FIG. 4, the two motors 52 are provided in the grasping section 6a such that the respective turning shafts 52b are parallel or substantially parallel to the longitudinal axis OX. The two motors 52 are disposed side by side along a plane including the tilting direction (the direction indicated by the arrow UDA) of the joystick 14 in order to bend the bending section 12 in the up-down direction.

Therefore, according to the embodiment explained above, it is possible to realize an endoscope with an improved grasping property of an operation section for endoscope that performs a bending operation using actuators.

In the present embodiment, since the two pulleys 61 are disposed on the same axis, an outer diameter of the respective pulleys 61 can be increased in a possible range in the operation main body section 6b.

Next, modifications are explained.

In the respective modifications explained below, a configuration of an entire endoscope apparatus is the same as the configuration in the embodiment explained above. Therefore, the same components as the components in the embodiment explained above are denoted by the same reference numerals and signs and explanation of the components is omitted. Different components are mainly explained.

### Modification 1

In the embodiment explained above, the two pulleys are disposed turnably on the same axis. However, in a modification 1, the two pulleys turn around axes different from each other.

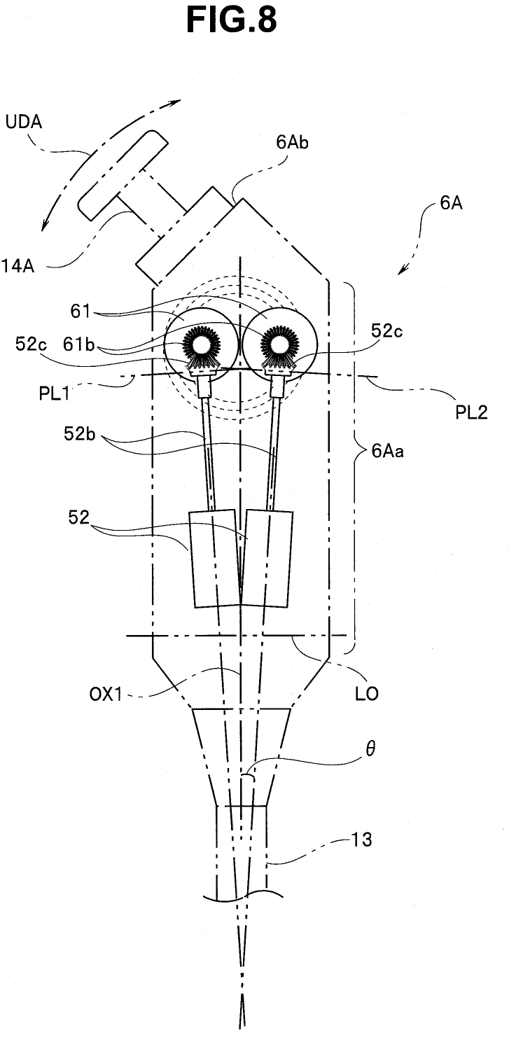

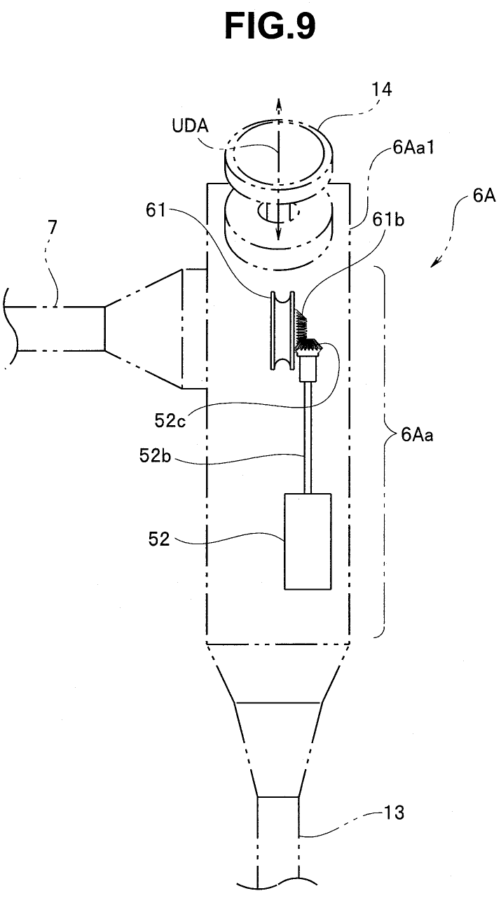

FIGS. 8 and 9 are schematic configuration diagrams of the operation section 6 showing disposition of actuators in the operation section 6 according to the modification 1. In FIGS. 8 and 9, only two motors 52 and two pulleys 62 are indicated by solid lines. An exterior member, a joystick, and the like other than the two motors 52 and the two pulleys 62 are indicated by alternate long and two short dashes lines.

An operation section 6A shown in FIGS. 8 and 9 has a shape of a so-called grip type including an elongated rectangular parallelepiped grasping section 6Aa. A proximal end of a flexible tube section 13 is connected to a distal end side (a lower side in FIG. 8) of the grasping section 6Aa. The connection cord 7 extends from a side surface of the grasping section 6Aa.

FIG. 8 is a diagram of the operation section 6A viewed from a palm side of the right hand when the surgeon grasps the grasping section 6Aa with, for example, the right hand. FIG. 9 is a diagram of the operation section 6A viewed from a joystick 14A side with respect to a longitudinal axis OX1 of the grasping section 6Aa when the surgeon grasps the grasping section 6Aa with, for example, the right hand. The surgeon can operate the joystick 14A with a thumb of the right hand while grasping the grasping section 6Aa with the right hand.

The joystick 14A is disposed in a slope section 6Ab formed on a proximal end side (an upper side in FIG. 8) of the grasping section 6Aa.

As shown in FIGS. 8 and 9, the two motors 52 are disposed side by side along a side surface 6Aa1 of the grasping section 6Aa in the grasping section 6Aa. A cross section of the grasping section 6Aa orthogonal to the longitudinal axis OX1 of the grasping section 6Aa has a substantially rectangular shape. Accordingly, the two motors 52 are disposed in the longitudinal axis LO direction of the grasping section 6Aa, the cross section of which is substantially rectangular.

Further, the turning shafts 52b extending from the respective motors 52 tilt by a predetermined angle θ with respect to the longitudinal axis OX1 such that an interval of the two bevel gears 52c is larger than an interval of the two motors 52.

At this time, as shown in FIG. 8, a plane PL1 orthogonal to one turning shaft 52b of the two turning shafts 52b and a plane PL2 orthogonal to the other turning shaft 52b of the two turning shafts 52b are not parallel, and cross at an angle 2θ.

In this way, each of the two motors 52 are disposed such that the turning shaft 52b tilts by the angle θ with respect to the longitudinal axis OX1. Consequently, it is possible to bring the two motors 52 close to each other. As a result, it is possible to effectively use a space in the grasping section 6Aa.

According to the modification 1 as well, since the two motors 52 having a relatively large weight are disposed in the longitudinal axis LO direction in the grasping section 6Aa, the cross section of which is rectangular, the grasping section 6Aa does not increase in size and the user can easily grasp the operation section 6A. As a result, the operability of the operation section 6A is high for the surgeon.

### Modification 2

The operation section 6A in the modification 1 explained above has the shape of the so-called grip type. However, an operation section in a modification 2 has a shape of a so-called gun grip type.

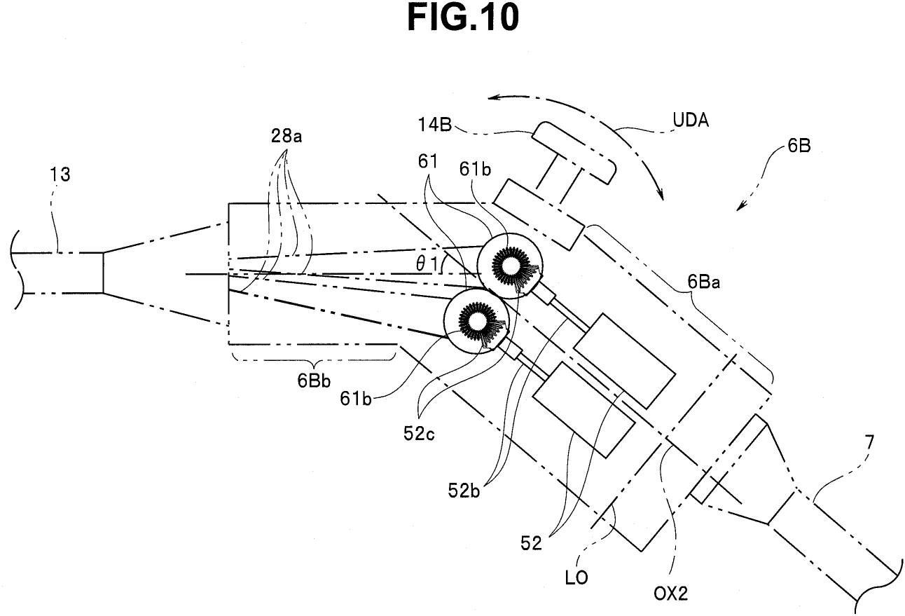

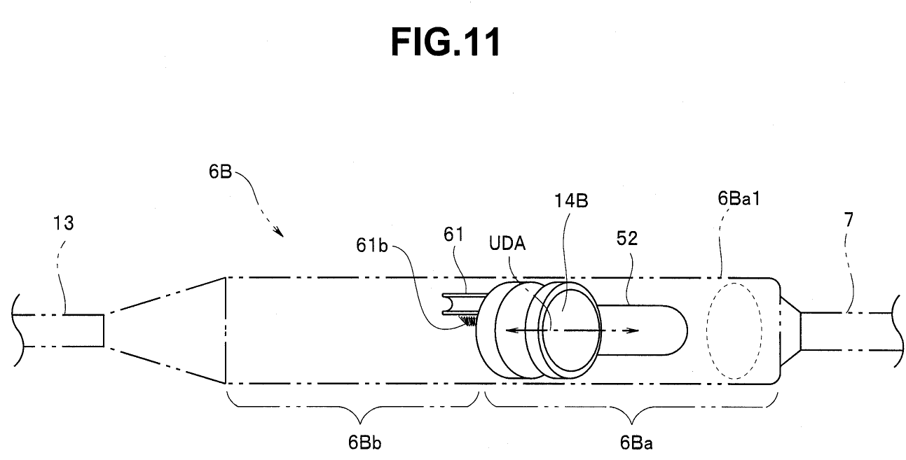

FIGS. 10 and 11 are schematic configuration diagrams of the operation section showing disposition of actuators in the operation section according to the modification 2. In FIGS. 10 and 11, only two motors 52 and two pulleys 62 are indicated by solid lines. An exterior member, a joystick, and the like other than the two motors 52 and the two pulleys 62 are indicated by alternate long two short dashes lines.

An operation section 6B shown in FIGS. 10 and 11 includes an elongated rectangular parallelepiped grasping section 6Ba. The grasping section 6Ba includes, on a distal end side, an extending section 6Bb extending while tilting by an angle θ1 with respect to a longitudinal axis OX2 of the grasping section 6Ba. The proximal end of the flexible tube section 13 is connected to a distal end side (a left side in FIG. 10) of the extending section 6Bb. The connection cord 7 extends from a proximal end of the grasping section 6Ba.

FIG. 10 is a diagram of the operation section 6B viewed from a side facing the palm of the right hand when the surgeon grasps the grasping section 6Ba with, for example, the right hand. FIG. 11 is a diagram of the operation section 6B viewed from a joystick 14B side with respect to a longitudinal axis OX2 of the grasping section 6Ba when the surgeon grasps the grasping section 6Ba with, for example, the right hand. The surgeon can operate the joystick 14B with the thumb of the right hand while grasping the grasping section 6Ba with the right hand.

The joystick 14B is disposed on a surface portion on a distal end side (an upper side in FIG. 10) of the grasping section 6Ba. In other words, a bending operation unit including the joystick 14B is disposed at an end portion on the insertion section 5 side of the grasping section 6Ba.

As shown in FIGS. 10 and 11, the two motors 52 are disposed side by side along a side surface 6Ba1 of the grasping section 6Ba in the grasping section 6Ba. A cross section of the grasping section 6Ba orthogonal to the longitudinal axis OX2 direction of the grasping section 6Ba has a substantially rectangular shape. Accordingly, the two motors 52 are disposed in the longitudinal axis LO direction of the grasping section 6Ba, the cross section of which is substantially rectangular.

In FIG. 10, the two motors 52 are disposed in the grasping section 6Ba such that the extending two turning shafts 52b are parallel.

Note that, in the modification 2, as in the modification 1, the respective motors 52 may be disposed to tilt, by the angle θ with respect to the longitudinal axis OX2, the two turning shafts 52b extending from the two motors 52 such that the interval of the two bevel gears 52c is larger than the interval of the two motors 52.

According to the modification 2 as well, since the two motors 52 having a relatively large weight are disposed side by side in a direction of a long diameter in a cross section orthogonal to the longitudinal axis OX2 of the rectangular grasping section 6Ba in the grasping section 6Ba, the grasping section 6Ba does not increase in size and the user can easily grasp the grasping section 6Ba. As a result, the operability of the operation section 6B is high for the surgeon.

As explained above, according to the embodiment and the respective modifications explained above, it is possible to provide an endoscope with an improved grasping property of an operation section for endoscope that performs a bending operation using actuators.

Note that, in the embodiment and the respective modifications explained above, the operation section includes the joystick as the operation member for performing the bending operation. However, the operation member does not have to be the joystick. For example, the operation member may be a disk-like knob turnable around an axis or a cross key tiltable in upward, downward, left, and right directions.

Furthermore, according to the embodiment and the respective modifications explained above, the wires 28a, which are the strip-shaped members, are laid in the pulley grooves on the outer circumferences of the pulleys 61. However, gears or sprockets may be used instead of the pulleys. Roller chains meshing with the gears or the sprockets may be used instead of the wires.

In the embodiment and the respective modifications explained above, the endoscope 2 includes the bending section 12 bendable in the upward, downward, left, and right directions. One of the two motors 52 is for up-down direction bending and the other is for left-right direction bending. However, in the case of the endoscope 2 having a configuration in which the bending section 12 is bendable only in the up-down direction, one of the two motors 52 may be for upward direction bending and the other of the two motors 52 may be downward direction bending.

In other words, in the embodiment and the respective modifications explained above, the bending section 12 is bendable in the upward, downward, left, and right four directions. However, the bending section 12 may be bendable only in upward and downward two directions.

In that case, one of the two motors 52 tows or slacks the wires 28a and 28 for upward direction bending via the turning shaft 52b and a bevel gear 2c. The other of the two motors 52 tows or slacks the wires 28a and 28 for downward direction bending via the turning shaft 52b and the bevel gear 2c.

For example, the processor 3a controls the turning of the two motors 52 not to simultaneously pull the wires 28a and 28 for upward direction bending and downward direction bending and, when the wires 28a and 28 for upward direction bending are pulled, slack the wires 28a and 28 for downward direction bending.

The present invention is not limited to the embodiment explained above. Various changes, alterations, and the like are possible within a range not changing the gist of the invention.

What is claimed is:

1. An endoscope comprising:
an elongated insertion section including a bending section configured to bend in at least an up-down direction by internally-disposed two or more strip-shaped members being towed;
an operation section including a grasping section grasped by a hand, the operation section being disposed on a proximal end side of the insertion section;
a bending operation unit including an operation member for bending the bending section and provided in the operation section;
two actuators each including turning shafts, the two turning shafts being internally disposed in the grasping section in a longitudinal direction of the grasping section, the two actuators being disposed side by side along a plane parallel to an operation direction of the operation member for bending the bending section in the up-down direction; and
a conversion mechanism including a rotation torque transmission mechanism to which rotation torque of the two turning shafts is transmitted and a tensile force transmission mechanism configured to transmit a tensile force for pulling the two or more strip-shaped members, the conversion mechanism converting the rotation torque into the tensile force.

2. The endoscope according to claim 1, wherein in the operation section, the rotation torque transmission mechanism is disposed on a bending operation unit side.

3. The endoscope according to claim 1, wherein
a first actuator of the two actuators transmits the rotation torque to a first torque conversion mechanism of the conversion mechanism, and
a second actuator of the two actuators transmits the rotation torque to a second torque conversion mechanism of the conversion mechanism.

4. The endoscope according to claim 3, wherein
the first conversion mechanism includes a first turning member configured to turn around a first turning shaft of the two turning shafts,
the second conversion mechanism includes a second turning member configured to turn around a second turning shaft of the two turning shafts, and
a first plane orthogonal to the first turning shaft and a second plane orthogonal to the second turning shaft cross at a predetermined angle.

5. The endoscope according to claim 1, wherein the bending operation unit is disposed at an end portion on an insertion section side of the grasping section.

6. The endoscope according to claim 1, wherein in a sectional shape orthogonal to a longitudinal direction of the grasping section, a dimension in a direction parallel to the plane is larger than a dimension in a direction orthogonal to the plane.

7. The endoscope according to claim 1, wherein
the two or more strip-shaped members are two or more wires, and
the conversion mechanism includes two pulleys on which the two or more wires are laid, the two pulleys pulling the two or more wires with the tensile force.

8. The endoscope according to claim 1, wherein the operation member is a joystick.
